SEQUENCE LISTING (8 sequences)

SEQ ID NO: 1 - DNA, 2261 bp, Homo sapiens
atctttgctgcaaaggctgggtatcggctgtgctcagcaaagcgtcaactcgtgcaagaacttagcaggaatagttctggctaaggttaggaggctgccaccaaagtctcttttttgttcctctgcttctcccgtttgcctccttatcatgagatctttttgctaagctggcagaaagattgcatagtcagtgcttccagctctgctcccacctgatcctgcactgtcctctggtccctgaatgaatgaactctgatacccaatcttgtctcgagccttctctatgccactcatggctcctcttctgctctttccatctttttgctgagagttctgagctctgtacttcctcttggcccatctcacttcctgaaacacccctgaagagggttgcttatcttgatggaactcaaaaagccaaaaagctgcaggcagaggcgttgaggacatctgtttggggaactaagagcagcagcactttcagattcagtccatatagagctgtcctacagcattctggaaacttgaggatgtgcggtgcataaaggggctggaagtgacccacctgtgatgagccctttctaaggagaagggtttccaagagatcaccccaccagaaaagggtaggaatgagcaagttgggaattttagactgtcactgcacatggacctctgggaagacgtctggcgagagctaggcccactggccctacagacggatcttgctggctcacctgtccctgtggaggttcccctgggaaggcaagatgcccaacaacagcactgctctgtcattggccaatgttacctacatcaccatggaaattttcattggactctgcgccatagtgggcaacgtgctggtcatctgcgtggtcaagctgaaccccagcctgcagaccaccaccttctatttcattgtctctctagccctggctgacattgctgttggggtgctggtcatgcctttggccattgttgtcagcctgggcatcacaatccacttctacagctgcctttttatgacttgcctactgcttatctttacccacgcctccatcatgtccttgctggccatcgctgtggaccgatacttgcgggtcaagcttaccgtcagatacaagagggtcaccactcacagaagaatatggctggccctgggcctttgctggctggtgtcattcctggtgggattgacccccatgtttggctggaacatgaaactgacctcagagtaccacagaaatgtcaccttcctttcatgccaatttgtttccgtcatgagaatggactacatggtatacttcagcttcctcacctggattttcatccccctggttgtcatgtgcgccatctatcttgacatcttttacatcattcggaacaaactcagtctgaacttatctaactccaaagagacaggtgcattttatggacgggagttcaagacggctaagtccttgtttctggttcttttcttgtttgctctgtcatggctgcctttatctatcatcaactgcatcatctactttaatggtgaggtaccacagcttgtgctgtacatgggcatcctgctgtcccatgccaactccatgatgaaccctatcgtctatgcctataaaataaagaagttcaaggaaacctaccttttgatcctcaaagcttgtgtggtctgccatccctctgattctttggacacaagcattgagaagaattctgagtagttatccatcagagatgactctgtctcattgaccttcagattccccatcaacaaacacttgagggcctgtatgcctgggccaagggatttttacatccttgattacttccactgaggtgggagcatctccagtgctccccaattatatctcccccactccactactctcttcctccacttcatttttcccttgtcctttctctctaattcagtgttttggaggcctgacttggggacaacgtattattgatattattgtctgttttccttcttcccaatagaagaataagtcatggagcctgaagggtgcctagttgacttactgacaaaaggctccagttgggctgaacatgtgtgtggtggtgactcatttccataccattgtggaattgagcagagaacctgctctcggaggatgcctaggagatgttgggaacagaaaaaataaactgagtttaagggggacttaaactgctgaattcacctgtggatgtttttgagtaaataaaagctaatagctaaaaaaaaaaaaaaaaaa

SEQ ID NO: 2 - protein, 318 aa, Homo sapiens
MPNNSTALSLANVTYITMEIFIGLCAIVGNVLVICVVKLNPSLQTTTFYFIVSLALADIAVGVLVMPLAIVVSLGITIHFYSCLFMTCLLLIFTHASIMSLLAIAVDRYLRVKLTVRYKRVTTHRRIWLALGLCWLVSFLVGLTPMFGWNMKLTSEYHRNVTFLSCQFVSVMRMDYMVYFSFLTWIFIPLVVMCAIYLDIFYIIRNKLSLNLSNSKETGAFYGREFKTAKSLFLVLFLFALSWLPLSIINCIIYFNGEVPQLVLYMGILLSHANSMMNPIVYAYKIKKFKETYLLILKACVVCHPSDSLDTSIEKNSE

SEQ ID NO: 3 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 4 - DNA, 2590 bp, Homo sapiens
acagcctgtgtacatgtgagcagccgcgtgcagaggggaagctctgcctgggcctcagggactgtgacatggagcaggagccgcccccagccaagctgctttcagcacagcgtgggcccccagcaccttggtgcggggtgcggcccctcggaggagggctgtcaggtgaagcctcgtgtgagggggtgcctcaggaaccctgaagctgggctgagccatgatgctgctgccagaacccctgcagagggcctggtttcaggagactcagagtcctctgtgaaaaagcccttggagagcgccccagcagggctgcacttggctcctgtgaggaaggggctcaggggtctgggcccctccgcctgggccgggctgggagccaggcgggcggctgggctgcagcaatggaccgtgagctggcccagcccgcgtccgtgctgagcctgcctgtcgtctgtggccatgcccatcatgggctcctcggtgtacatcacggtggagctggccattgctgtgctggccatcctgggcaatgtgctggtgtgctgggccgtgtggctcaacagcaacctgcagaacgtcaccaactactttgtggtgtcactggcggcggccgacatcgcagtgggtgtgctcgccatcccctttgccatcaccatcagcaccgggttctgcgctgcctgccacggctgcctcttcattgcctgcttcgtcctggtcctcacgcagagctccatcttcagtctcctggccatcgccattgaccgctacattgccatccgcatcccgctccggtacaatggcttggtgaccggcacgagggctaagggcatcattgccatctgctgggtgctgtcgtttgccatcggcctgactcccatgctaggttggaacaactgcggtcagccaaaggagggcaagaaccactcccagggctgcggggagggccaagtggcctgtctctttgaggatgtggtccccatgaactacatggtgtacttcaacttctttgcctgtgtgctggtgcccctgctgctcatgctgggtgtctatttgcggatcttcctggcggcgcgacgacagctgaagcagatggagagccagcctctgccgggggagcgggcacggtccacactgcagaaggaggtccatgctgccaagtcactggccatcattgtggggctctttgccctctgctggctgcccctacacatcatcaactgcttcactttcttctgccccgactgcagccacgcccctctctggctcatgtacctggccatcgtcctctcccacaccaattcggttgtgaatcccttcatctacgcctaccgtatccgcgagttccgccagaccttccgcaagatcattcgcagccacgtcctgaggcagcaagaacctttcaaggcagctggcaccagtgcccgggtcttggcagctcatggcagtgacggagagcaggtcagcctccgtctcaacggccacccgccaggagtgtgggccaacggcagtgctccccaccctgagcggaggcccaatggctatgccctggggctggtgagtggagggagtgcccaagagtcccaggggaacacgggcctcccagacgtggagctccttagccatgagctcaagggagtgtgcccagagccccctggcctagatgaccccctggcccaggatggagcaggagtgtcctgatgattcatggagtttgccccttcctaagggaaggagatctttatctttctggttggcttgaccagtcacgttgggagaagagagagagtgccaggagaccctgagggcagccggttcctactttggactgagagaagggagccccaggctggagcagcatgaggcccagcaagaagggcttgggttctgaggaagcagatgtttcatgctgtgaggccttgcaccaggtgggggccacagcaccagcagcatctttgctgggcagggcccagccctccactgcagaagcatctggaagcaccaccttgtctccacagagcagcttgggcacagcagactggcctggccctgagactggggagtggctccaacagcctcctgccacccacacaccactctccctagactctcctagggttcaggagctgctgggcccagaggtgacatttgacttttttccaggaaaaatgtaagtgtgaggaaaccctttttattttattacctttcactctctggctgctgggtctgccgtcggtcctgctgctaacctggcaccagagcctctgcccggggagcctcaggcagtcctctcctgctgtcacagctgccatccacttctcagtcccagggccatctcttggagtgacaaagctgggatcaaggacagggagttgtaacagagcagtgccagagcatgggcccaggtcccaggggagaggttggggctggcaggccactggcatgtgctgagtagcgcagagctacccagtgagaggccttgtctaactgcctttccttctaaagggaatgtttttttctgagataaaataaaaacgagccacatcgtgttttaagcttgtccaaatgaaaaaaaaaaaaaaaaaaaaaaaaa

SEQ ID NO: 5 - protein, 412 aa, Homo sapiens
MPIMGSSVYITVELAIAVLAILGNVLVCWAVWLNSNLQNVTNYFVVSLAAADIAVGVLAIPFAITISTGFCAACHGCLFIACFVLVLTQSSIFSLLAIAIDRYIAIRIPLRYNGLVTGTRAKGIIAICWVLSFAIGLTPMLGWNNCGQPKEGKNHSQGCGEGQVACLFEDVVPMNYMVYFNFFACVLVPLLLMLGVYLRIFLAARRQLKQMESQPLPGERARSTLQKEVHAAKSLAIIVGLFALCWLPLHIINCFTFFCPDCSHAPLWLMYLAIVLSHTNSVVNPFIYAYRIREFRQTFRKIIRSHVLRQQEPFKAAGTSARVLAAHGSDGEQVSLRLNGHPPGVWANGSAPHPERRPNGYALGLVSGGSAQESQGNTGLPDVELLSHELKGVCPEPPGLDDPLAQDGAGVS

SEQ ID NO: 6 - DNA, 21 bp, Homo sapiens
caggagtgtcctgatgattca

SEQ ID NO: 7 - DNA, 21 bp, Homo sapiens
cacgtatctagctaatatgta

SEQ ID NO: 8 - DNA, 21 bp, Homo sapiens
ccctatcgtctatgcctataa

(19) United States
(12) Patent Application Publication
(10) Pub. No.: US 2010/0105758 A1
(43) Pub. Date: Apr. 29, 2010

(54) USE OF AN ADENOSINE ANTAGONIST

(75) Inventors: Daniel R. Wagner, Bertrange (LU); Yvan Devaux, Flavigny/Moselle (FR)

Correspondence Address: QUINE INTELLECTUAL PROPERTY LAW GROUP, P.C., P O BOX 458, ALAMEDA, CA, 94501, US

(73) Assignee: Centre de Recherche Public de la Sante, Luxembourg (LU)

(21) Appl. No.: 12/312,410

(22) PCT Filed: Nov. 8, 2007
(86) PCT No.: PCT/EP2007/009932

§ 371 Date: Oct. 7, 2009

Related U.S. Application Data
(60) Provisional application No. 60/858,267, filed on Nov. 9, 2006.

(51) Int. Cl. A61K 31/7088; A61K 31/4418; A61K 31/4375; A61K 31/519; A61P 9/00; A61K 31/7076

(52) U.S. Cl. 514/44 A; 514/356; 514/294; 514/267; 514/46

(57) ABSTRACT

Uses for a selective adenosine A3 receptor antagonist, or RNAi directed against said receptor, to treat myocardial infarction and heart conditions including heart failure, are provided. Optionally, an adenosine A2a receptor agonist may also be used with the adenosine A3 receptor antagonist. Methods of treating heart failure are also provided.

This application claims priority from U.S. provisional application U.S. 60/858,267, which is hereby incorporated by reference.

### FIELD OF THE INVENTION

The present invention relates to the use of a selective adenosine A3 receptor antagonist, or RNAi directed against said receptor, to treat myocardial infarction and various heart conditions including heart failure.

### BACKGROUND OF THE INVENTION

Congestive heart failure (CHF) is a compilation of signs and symptoms, all of which are caused by an inability of the heart to appropriately increase cardiac output as needed. Patients typically present with shortness of breath, edema and fatigue. CHF has become a disease of epidemic proportion, affecting 3% of the adult population. Mortality of CHF is worse than many forms of cancer with a five year survival of less than 30%. Myocardial infarction (MI) is one of the leading causes of CHF. Left ventricular (LV) remodelling contributes largely to CHF.

It is now well recognized that changes in the myocardial extracellular matrix (ECM) contribute to the progressive remodelling process. A balance of ECM synthesis and degradation, also called ECM turnover, determines the maintenance of tissue architecture. The normal rate of ECM synthesis in the heart is very low. During pathological situations, such as MI, collagen synthesis and deposition is accelerated not only in the infarcted, but also in the non-infarcted myocardium. Growing evidence suggests that changes in fibrillar collagen network and collagen matrix disorganization contribute to LV remodelling.

Matrix disorganization has been attributed to increased expression of matrix metalloproteinases (MMPs), which break down matrix proteins and decrease expression of tissue inhibitors of metalloproteinase (TIMPs), a family of protease inhibitors. MMPs are important molecules that are the driving force behind the degradation of the myocardial ECM. Recent studies have clearly demonstrated that in the heart, MMPs contribute to ventricular remodelling and heart failure. At the clinical level, studies from our group and by others have shown that elevated blood levels of MMPs are associated with the development of heart failure after MI. Therefore, measurement of MMPs, and in particular MMP-9, in patients with MI or CHF provides a prognostic measure similar to that of TNF-α, angiotensin II or norepinephrine.

A study with MMP-9 deficient mice revealed MMP-9 as a potential therapeutic target to prevent LV remodelling after MI. However, the PREMIER (Prevention of Myocardial Infarction Early Remodelling) phase II trial showed that non-specific inhibition of MMPs activity with PG-116800 failed to prevent LV remodelling and did not improve outcome after MI. In this study, the MMP inhibitor was given 24-72 hours post MI. The negative results of this trial may in part be explained by the non-specificity of the MMP inhibitor and suggest that further experimental work is necessary to understand the implication of MMPs in LV remodelling.

Analogous to neurohormones and pro-inflammatory cytokines such as tumour necrosis factor-α (TNF-α), MMPs therefore represent another distinct class of biologically active molecules that can contribute to heart failure progression. There is a growing body of evidence that suggests that modulating inflammatory cytokines and MMP levels may represent a new therapeutic paradigm for treating patients with heart failure.

Polymorphonuclear leukocytes (neutrophils or PMNs) and macrophages are rich sources of MMPs. Neutrophils are among the first wave of cells recruited to infarcted tissues. The second wave of inflammatory cells recruited to the infarcted tissues consists mainly of monocytes/macrophages. Among these, macrophages are the major contributor of MMP-9 secretion. Recruitment and activation of monocytes/macrophages in the infarcted myocardium has been shown to contribute importantly to the processes that occur after MI. These cells migrate along a gradient of the chemokine Monocyte Chemoattractant Protein (MCP)-1, which binds to its receptor CC chemokine receptor-2 (CCR2) expressed at the cell surface.

It has only recently been recognized that macrophages play an important role in the remodelling process of the heart. This is indicated by the fact that expression of MCP-1 and infiltration of the myocardium with macrophages is increased in post MI hearts and in the failing human and animal heart. It is known that MCP-1 is mediating the recruitment of macrophages into the myocardial tissue. In addition, transgenic cardiac overexpression of MCP-1 results in cardiac remodelling and heart failure. On the other hand, however, inhibition of MCP-1 signalling has been shown to attenuate progressive cardiac dysfunction in a murine model of MI.

Cells of the cardiovascular system produce adenosine (ado), a purine nucleoside, in conditions of stress and injury. Adenosine has been thought for long time to be cardioprotective in the setting of myocardial ischaemia. To date, four adenosine receptor subtypes have been characterized: A1, A2a, A2b, and A3. All four subtypes appear to be expressed within the cardiovascular system. Adenosine released during ischaemia results in effective preconditioning in cardiomyocytes.

The adenosine A2a receptor is involved in vasodilatation of the aorta and the coronary artery. In the late 1960s and 1970s, adenosine A2 agonists were tested clinically as anti-hypertensives but abandoned because of poor in-vivo selectivity. In platelets, adenosine A2 agonists have been shown to inhibit platelet aggregation by increasing intracellular cAMP levels. Adenosine A2 agonists have also been developed for myocardial stress imaging to evaluate coronary artery disease by achieving vasodilatation in patients unable to exercise on the bike or treadmill. Regadenoson (CVT-3146), a selective adenosine A2 agonist, is currently being evaluated in Phase III studies for myocardial perfusion imaging.

Adenosine A2a agonists have been shown to attenuate inflammation and reperfusion injury in several tissues. The adenosine A2a receptor is expressed in nearly all immune cells including neutrophils and macrophages and this receptor has been referred to as a “brake for inflammation.” Indeed, adenosine A2a knockout mice show that this receptor is essential to limit inflammation. We have previously shown that adenosine A2a receptor agonists inhibit the production of TNF-α in cardiac cells. Adenosine A2A agonists are currently being evaluated for the treatment of sepsis, inflammatory bowel disease and wound healing. Because of interaction between the adenosine A2a receptor and dopamine D receptors, adenosine A2a antagonists are currently being evaluated in Parkinson's disease. Adenosine A2b receptor agonists have been shown to inhibit cardiac fibroblasts and to promote angiogenesis.

The therapeutic potential of adenosine or adenosine analogues has been reported in both animal studies and clinical trials. In animals, adenosine's cardioprotective activities were found during the three windows of potential therapeutic action for ischaemia-reperfusion: as a pre-treatment, during ischaemia or during reperfusion. A dichotomy between the types of receptors involved was first hypothesized, with A1 receptors being proposed as mediating the cardioprotective effects of adenosine during pre-treatment and during ischaemia, mainly through metabolic changes, and A2 receptors being beneficial during reperfusion, mainly through inhibition of neutrophil activity. Our previous data support the involvement of A2a receptors in the protection afforded by adenosine during post-ischaemic injury, through inhibition of MMP-9 release by neutrophils (Ernens et al. “Adenosine inhibits matrix metalloproteinase-9 secretion by neutrophils: implication of A2a receptor and cAMP/PKA/Ca2+ pathway.” Circ Res. 2006; 99(6):590-597, incorporated herein by reference).

Adenosine A3 receptor activation has been found cardioprotective before or during ischaemia, through neutrophil-dependent as well as neutrophil-independent mechanisms.

Few clinical trials have tested the therapeutic potential of adenosine (ado) in the context of MI. Adenosine improved cardiac function of patients with MI when added in combination with lidocaine or primary angioplasty. However, these trials, although reporting a beneficial clinical outcome, were performed on a small number of patients. The AMISTAD I and II trials, performed on larger sample size (236 and 2,118 patients, respectively), demonstrated a reduction in infarct size by adenosine as an adjunct to reperfusion therapy, when added early after infarction. A post-hoc analysis of the AMISTAD-II trial revealed that adenosine reduced mortality only when added within 3 hours of infarction. Of note, in this trial adenosine was administered for more than 48 hours after MI.

Thus, there is a need in the art for further treatments for congestive heart failure, particularly with a view to improving survival rates and lessening the development of worsening heart failure for these often fatal heart conditions or diseases.

There is a large volume of prior art on the subject of the use of adenosine or adenosine receptor agonists. For instance, we showed in Ernens et al. that adenosine reduces the secretion of MMP-9 in neutrophils through the A2a receptor. Thus, the art points towards the use of adenosine in many, if not all, instances of heart failure.

Surprisingly, however, we have now shown that adenosine can both stimulate and inhibit the release of MMP-9 depending on the cell type and the type of receptor involved. In fact, in contrast to the previous art, including our own findings, we have found that adenosine actually induces the secretion of MMP-9 by monocytes/macrophages through the adenosine A3 receptor.

### SUMMARY OF THE INVENTION

Thus, in first aspect, the present invention provides for the use of an adenosine A3 receptor antagonist in the treatment or prophylaxis of a disease or condition associated with congestive heart failure.

Preferably, the adenosine A3 receptor antagonist is used to treat a patient with myocardial infarction or heart failure (including acute heart failure or chronic heart failure). It is also preferred that the use of an adenosine A3 receptor antagonist decreases levels of matrix metalloproteinases in a patient with myocardial infarction or heart failure.

Preferably, the adenosine A3 receptor antagonist is used to prevent the development of ventricular remodelling and heart failure after myocardial infarction. In particular, this is to prevent or reduce maladaptive remodelling of the myocardium. This may include fibrosis, apoptosis and necrosis.

Preferably, the adenosine A3 receptor antagonist is a polypeptide or protein, or a polynucleotide encoding it, the polynucleotide being preferably administered in a vector with a suitable promoter operably linked to the polynucleotide.

The adenosine A3 receptor antagonist is preferably a nucleoside, but may also be a non-nucleoside inhibitor. Suitable examples of the adenosine A3 receptor antagonist are MRS 1067, MRS 1097, L-249313, L-268605, CGS15943, KF26777. Particularly preferred are MRS1220, MRS1523, PSB10. MRS1220, MRS1523 can be purchased from Sigma-Aldrich. PSB10 can be purchased from Tocris.

It is also preferred that further pharmaceutically active ingredients or polypeptides or polynucleotides having effector functions may be administered together with the adenosine A3 receptor antagonist. These are preferably further adenosine agonists or antagonists and most preferably an adenosine A2a receptor agonist.

Preferably, the adenosine A3 receptor antagonist also has further adenosine agonist or antagonist activity. Particularly preferred is a molecule having both A3 receptor antagonist activity and A2a receptor agonist activity, for example (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol.

The nucleotide sequence encoding the adenosine A3 receptor is preferably that given in NO 1, but may or may not include the polyA tail for instance. The protein sequence is preferably that provided in SEQ ID NO 2, although post-translational modification, particularly removal of the N-term Met is envisaged.

It is preferred that the adenosine A3 receptor antagonist is capable of reducing the levels of an MMP in the blood or in cardiac tissue, especially in and around the heart, and particularly around any infarcted or ischaemic tissue. The levels may be decreased by reducing expression of the adenosine A3 receptor or by binding thereto, preferably in a non-permanent competitive manner, thereby temporarily blocking the ability of adenosine or an adenosine analogue or adenosine agonist from binding to, and thereby stimulating, the adenosine A3 receptor to initiate release, or preferably secretion, of the MMP from the cell. Therefore, it is envisaged that the adenosine A3 receptor antagonist is preferably capable of reducing secretion of MMP from the cell. The cell is preferably a monocyte and most preferably a macrophage, although other cells bearing the adenosine A3 receptor are also envisaged, in a particular embodiment.

The A3 antagonist is preferably selective. Even more preferably, the antagonist is specific for the A3 receptor. A compound or molecule may be considered a specific or at least selective antagonist of the A3 receptor site if the compound binds the A3 receptor with a higher affinity than adenosine, with preferably at least 5 times and more preferably at least 10 times greater affinity than adenosine.

The effect of the A3 antagonist is preferably reversible and not irreversible. The antagonist may be irreversible, but this is not preferred in a clinical setting as this could lead to permanent inhibition of the A3 receptor and hence permanent inhibition of MMP (especially MMP-9) levels in the patient.

The above also to any further agonists or antagonists additionally provided, which may also be selective or specific and may also be preferably reversible.

The MMP is, most preferably, MMP-9, although other MMPs are envisaged in alternative embodiments. MMP-9 preferably has the protein sequence set out in SEQ ID NO 3 although post-translational modification, particularly removal of the N-term Met is envisaged.

The invention also provides for the use of antisense polynucleotides, particularly antisenese RNA, such as interference RNA (RNAi) including microRNA (miR or miRNA) or short interfering RNA (siRNA) and other forms of gene suppression, targeted to the adenosine A3 receptor (A3AR) or capable of reducing the levels or expression thereof. When using an A3AR antagonist or the antisense polynucleotides, the effect is to reduce the adenosine-stimulated response mediated by the adenosine A3 receptor.

Preferably, the antisense polynucleotides are targeted to the sequence of the adenosine A3 receptor. The sequence of this receptor is preferably that given in SEQ ID NO 1. In the case of microRNA, the antisense polynucleotides preferably target the 3′UTR of the adenosine A3 receptor. Preferred target sequences for the antisense polynucleotides are provided in any of SEQ ID NOS 6, 7 or 8, with SEQ ID NO 8 being particularly preferred, as it is an A3 receptor sequence.

Suitable methods of administration, introduction or expression in vivo of the antisense polynucleotides are well known in the art, but may include direct administration of the antisense polynucleotides or expression of the antisense polynucleotides by a suitable vector. As above, administration may be orally, via a mucous membrane, transdermally, for instance via a suitable patch or gene-gun, sub-cutaneously, intra-muscularly or intravenously.

Also provided are methods of treating patients with heart failure or conditions associated therewith, comprising administering an adenosine A3 receptor antagonist to the patient. Preferably, an adenosine A2a receptor agonist may also be administered at the same time or after, but preferably before the adenosine A3 receptor antagonist. The nucleotide sequence encoding the adenosine A2a receptor is preferably that given in NO 3, but may or may not include the polyA tail, for instance. The protein sequence is preferably that provided in SEQ ID NO 4, although post-translational modification, particularly removal of the N-term Met is envisaged.

### BRIEF DESCRIPTION OF THE DRAWINGS

FIG. 1 shows that adenosine increases MMP-9 production by primary human macrophages, as assessed by ELISA in conditioned medium or by quantitative PCR in cells.

FIG. 2 shows that adenosine dose- and time-dependently increases MMP-9 production by THP-1-derived macrophages.

FIG. 3 shows that adenosine increases MMP-9 production by macrophages through its A3 receptor.

FIG. 4 shows that the adenosine-mediated increase of MMP-9 production facilitates monocytes migration through a gelatin matrix.

### BRIEF DESCRIPTION OF THE INVENTION

A3-mediated protection appears to be dose-dependent, as mice with mild over-expression of A3 were partly protected from ischaemic injury whereas mice which highly over-expressed A3 receptor developed a dilated cardiomyopathy. This is consistent with our observation that stimulation of the A3 receptor is associated with MMP-9 secretion and migration of macrophages.

The fact that adenosine prevents MMP-9 release by neutrophils whereas it enhances MM-9 secretion by macrophages has several explanations and biological meanings. Neutrophils are recruited to the infarct area early after MI (<2 days) whereas macrophages invade the lesion 2-4 days post MI. The mechanisms responsible for MMP-9 production by these two cell types are fundamentally different.

Neutrophils rapidly release large amounts of MMP-9 by degranulation. In contrast, MMP-9 secretion by macrophages is not detected before 8 hours, which is consistent with de novo protein synthesis. We previously demonstrated that in neutrophils adenosine signals through the A2a receptor to decrease MMP-9 secretion and this mechanism involved a quick signalling cascade with rapid Ca mobilization (See Ernens et al, supra). In contrast, we now show that enhancement of MMP-9 production by macrophages is mediated through A3 receptor activation and involves a transcriptional effect. Therefore, the effects of adenosine on MMP-9 production by inflammatory cells are highly dependent on the type of adenosine receptor and signalling pathway which are activated.

The use of adenosine in the treatment of various heart problems including infarction and ischaemia is well known. Indeed, the present inventors have published a paper in August 2006 (Ernens et al), which shows that adenosine reduces the release of MMP-9 by neutrophils via the A2a receptor. However, they have now found that adenosine also induces the secretion of MMP-9 by monocytes/macrophages via the A3 receptor. In other words, adenosine reduces MMP-9 levels (in the very early stages post-infarction, for instance) when neutrophils bearing the A2a receptor are present. However, after some time, monocytes and macrophages appear at the infarcted tissue, and these cells bear the A3 receptor. In the presence of the A3 receptor, adenosine has now been shown to increase the levels of MMP-9. Therefore, administration of adenosine has different effects on the levels of MMP-9 in the presence of the different receptors.

We have demonstrated that adenosine is not necessarily beneficial in the context of infarction and remodelling. This is in complete contradiction to the current paradigm that adenosine is a retaliatory metabolite and that adenosine is protective during periods of stress. The effect of adenosine on MMP-9 was mediated through the adenosine A2a receptor in neutrophils (inhibition) and through the adenosine A3 receptor in macrophages (stimulation). So far, the adenosine A2a receptor has been known to inhibit several immune processes including degranulation of polymorphonuclear leukocytes, production of oxygen free radicals and TNF-α. Thus, adenosine has been considered as a physiological brake that may limit organ damage through inflammation.

Our results indicate for the first time that the adenosine A3 receptor may lead to myocardial remodelling. Thus there is an intimate link between adenosine and stress, MMPs and pro-inflammatory cytokines, possibly representing a vicious circle implicated in myocardial failure.

Accordingly, we propose a totally novel approach of preventing and treating conditions or diseases associated with congestive heart failure, including myocardial infarction, through inhibition of the A3 receptor. Adenosine A3 antagonists could therefore be useful in patients with acute MI and heart failure to inhibit the release of MMPs, particularly by monocytes and macrophages. Furthermore, as the release of MMP-9 by macrophages is a driving force of left ventricular remodelling in acute myocardial infarction and heart failure, we conclude that adenosine A3 antagonists could be helpful to prevent left ventricular remodelling.

This is absolutely opposite to the approach previously taken in the art. For instance, Liang et al. (U.S. Pat. No. 6,211,165 B1) disclose an adenosine A2a receptor antagonist and an adenosine A3 receptor agonist, which is the opposite arrangement of agonist and antagonist according to an embodiment of the present invention.

It is also envisaged that the present invention can be used in combination with other treatments. These may be other drugs or active ingredients suitable for administration to the patient for treatment of their condition. However, it is particularly preferred that the further treatment comprises or is based upon the use of further adenosine agonists or antagonists. For instance, an adenosine A3 antagonist together with an adenosine A2a agonist could also be useful in patients with acute MI and heart failure to inhibit the release of MMPs by neutrophils (A2a) and macrophages (A3). Similarly, a combination of adenosine A3 antagonist with an adenosine A2a agonist could be helpful to prevent left ventricular remodelling. This is absolutely opposite to current beliefs.

Therefore, stimulation of the adenosine A2a receptor by an adenosine agonist or adenosine analogue is particularly preferred. Suitable adenosine agonists or adenosine analogues for the A2a receptor are well known in the art. Preferably, these include the A2a agonists Regadenoson (CVT-3146), CGS 21680, APEC and 2HE-NECA. Adenosine may also be used.

Although not preferred, a suitable A3 agonist, if required in addition to, or for later administration to, the A3 agonist is IB-MECA, if required. Although also not preferred, a suitable A2a antagonist is SCH58261.

Although the use of an adenosine A2a receptor agonist is particularly preferred in combination with the adenosine A3 receptor antagonist of the present invention, it is envisaged that other adenosine receptor agonists or antagonists may also be used.

Thus, it is preferred that a further adenosine agonist or antagonist is used together with the adenosine A3 receptor antagonist of the present invention. Preferably, this is a further adenosine agonist, which may be adenosine itself or an analogue thereof. Alternatively, a further adenosine antagonist may used.

The further adenosine agonist or antagonist may target a different adenosine receptor, or may have a different activity in terms of the strength and timing of the response that it induces. For instance, the further adenosine agonist or antagonist may be cleared at a different rate from the blood or may also have an additional therapeutic effect which could be beneficial to the patient. Preferably the receptor is the A1 receptor, more preferably the A2 receptor, of which the A2a receptor is particularly preferred. The A1b receptor is less preferred as it has a lower affinity for adenosine than the other receptors, but may be targeted where adenosine or is not used. The A3 receptor may also be targeted by another A3 antagonist.

Suitable adenosine receptor agonists or antagonists are known in the art and some examples are described herein.

Such stimulation by a further adenosine agonist or antagonist may be co-temporaneous or concomitant to the inhibition of the adenosine A3 receptor, or may at a different time. For instance, the adenosine A3 receptor antagonist may be administered first, followed by the further adenosine agonist or antagonist.

However, it is preferred that the adenosine A2a receptor agonist is administered within 8 hours of any infarction, as the monocytes and macrophages bearing the A3 receptor are not thought to generally arrive in the infarcted tissue until around that amount of time has lapsed, although this will vary and will require an accurate estimation of the time of infarction.

This may mean that the adenosine A2a receptor agonist is administered before the adenosine A3 receptor antagonist. Alternatively, they may be administered at around the same time.

It is also particularly preferred that the adenosine A3 receptor antagonist activity and any further agonist or antagonist activity are provided by the same compound. Alternatively, the adenosine A3 receptor antagonist activity and any further agonist or antagonist activity may be provided by linked or conjugated compounds, where one or more parts of the linked or conjugated compound has the adenosine A3 receptor antagonist activity, whilst another part has the further agonist or antagonist activity. In other words, this could be two normally separate or distinct molecules linked together, or this could be provided by a single molecule.

A preferred example of the latter is a molecule having both A3 receptor antagonist activity and A2a receptor agonist activity. Particularly preferred is (2R,3R,4S,5R)-2-(6-amino-2-{[(1S)-2-hydroxy-1-(phenylmethy)ethyl]amino}-9H-purin-9-yl)-5-(2-ethyl-2H-tetrazol-5-yl)tetrahydro-3,4-furandiol from GlaxoSmithKline, published in June 2007 (Eur J Pharmacol. 2007 Jun. 14; 564(1-3):219-25. Pharmacological characterisation and inhibitory effects of (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol, a novel ligand that demonstrates both adenosine A(2A) receptor agonist and adenosine A(3) receptor antagonist activity. Bevan N, Butchers P R, Cousins R, Coates J, Edgar E V, Morrison V, Sheehan M J, Reeves J, Wilson D J). This paper focuses on A2a agonist activity of this molecule in relation to neutrophils. The A3 antagonist activity is mentioned in relation to inhibition of generation of reactive oxygen species from eosinophils, but eosinophils are not part of the pathophysiological response of the heart to ischaemia and thus are not directly involved in the development of heart failure.

The nucleotide and protein sequences of both the A2a and the A3 receptors are preferably those available at NCBI accession numbers NM_{—}000675 (A2a) and NM_{—}000677 (A3). The protein sequence for MMP-9 is preferably NCBI accession number NM_{—}004994.

Where reference is made to an adenosine A3 antagonist, it will be appreciated that this could be one A3 antagonist, which is preferred, or at least one A3 antagonist (i.e. a mixture of 2 or more different A3 antagonists). The same applies mutatis mutandis to adenosine A2a agonists.

Reference to a particular sequence also preferably includes variants with a degree of sequence homology, whilst still retaining a reasonable degree of the functionality of the reference sequence. For nucleotide sequences, this is preferably a sequence having at least 70%, more preferably at least 75%, more preferably at least 80%, more preferably at least 85%, more preferably at least 90%, more preferably at least 95%, more preferably at least 99%, more preferably at least 99.5%, and most preferably at least 99.9% sequence homology with the reference sequence, as appropriate, which may be due to a number of mismatches, substitutions, insertions or deletions. Where the reference sequence is DNA, this includes the corresponding RNA sequence and visa versa. Indeed, sequences that are capable of binding to the reference sequence under highly stringent conditions, for instance 6×SSC, are also preferred. The term “hybridise under stringent conditions” means that two oligonucleotides are capable to hybridise with one another under standard hybridisation conditions as described in Sambrook, et al. Molecular Cloning: A laboratory manual (1989), Cold Spring Harbor Laboratory Press, New York, USA. For this purpose, it is also possible to use common stringent hybridization conditions (e.g. 60 DEG C., 0.1×SSC, 0.1% SDS), for example.

Where the reference sequence is a polypeptide sequence, the variant sequence preferably has at least 85%, more preferably at least 90%, more preferably at least 95%, more preferably at least 99%, more preferably at least 99.5%, and most preferably at least 99.9% sequence homology with the reference sequence, as appropriate.

Suitable methods for assessing sequence homology are known in the art and include the BLAST program.

Reference to treatment or prophylaxis may be interpreted as to improving the clinical outcome of the patient. Reference to preventing and treating conditions or diseases “associated with” congestive heart failure may be understood to include a disease or condition that directly causes or contributes to congestive heart failure. Several examples are provided herein, such as myocardial infarction, acute coronary syndrome, ischaemic cardiomyopathy, non-ischaemic cardiomyopathy, or acute or chronic heart failure and so forth.

The present invention also provides a method of treating a patient with myocardial infarction or heart failure, by administration of an adenosine A3 receptor antagonist and optionally a further adenosine agonist or adenosine antagonist. The further adenosine agonist or adenosine antagonist is preferably an adenosine A2a receptor agonist.

In a further aspect, the present invention provides a method of inhibiting ventricular remodelling.

In a still further aspect, the present invention provides a method for lowering or reducing MMP-9 levels in a patient's heart. In a related aspect, the present invention also provides a method for the treatment or prophylaxis of heart failure correlated with lower MMP-9 levels. Lower MMP-9 levels predict a better clinical outcome after myocardial infarction. These aspects of the invention may be preferably provided by inhibition of MMP-9 production by both neutrophils (through A2a receptor inhibition) and macrophages (through A3 receptor inhibition), by administration of an adenosine A3 receptor antagonist and an adenosine A2a receptor agonist.

Also provided is a method of preventing degradation of myocardial tissue associated with myocardial infarction or acute heart failure comprising administration of an adenosine A3 receptor antagonist and optionally a further adenosine agonist or adenosine antagonist. The further adenosine agonist or adenosine antagonist is preferably an adenosine A2a receptor agonist.

The present methods allow the treatment of a newly-diagnosed patient for the improvement of an existing therapeutic strategy of a patient, for instance following myocardial infarction.

The condition is preferably myocardial infarction, acute coronary syndrome, ischaemic cardiomyopathy, non-ischaemic cardiomyopathy, acute heart failure or congestive heart failure.

Reference to treatment also encompasses prophylaxis, where appropriate. Where reference is made to the use or administration of a particular active ingredient, this should be in a therapeutically effective amount.

Also provided is a method of preventing degradation of myocardial tissue associated with end-stage heart disease.

The invention also provides a method of treating a patient presenting symptoms of congestive heart failure comprising administering an agent which decreases the production of matrix metalloproteinases in the myocardial tissue. Preferably, the symptoms are indicative of acute heart failure and wherein said agent which decreases the production of matrix metalloproteinases in the myocardial tissue is comprised of a therapeutically effective A3 receptor antagonist and optionally an A2a agonist. Preferably, the symptoms are indicative of chronic heart failure and wherein said agent which decreases the production of matrix metalloproteinases in the myocardial tissue is comprised of a therapeutically effective A3 receptor antagonist and optionally an A2a agonist.

All references cited herein are hereby incorporated by reference. Specific embodiments of the invention will now be described with reference to the accompanying drawings and in the following Examples.

### EXAMPLES

### Introduction

Matrix metalloproteinases (MMPs), and in particular MMP-9, are very important compounds that are the driving force behind the degradation of the myocardial extracellular matrix. Recent studies have clearly demonstrated that in the heart, MMPs contribute to ventricular remodelling and heart failure. At the clinical level, studies from our group recently confirmed by others have shown that elevated blood levels of MMPs are associated with the development of heart failure after MI. Neutrophils and macrophages play an important role in the inflammatory responses that lead to myocardial damage and fibrosis, at least partly through production of large quantities of MMP-9.

Cardioprotective properties of the nucleoside adenosine are known. Its potential therapeutic use in the context of myocardial infarction and heart failure deserves consideration. Four adenosine receptors have been characterized: A1, A2a, A2b, A3. We have previously shown that adenosine inhibits MMP-9 secretion by neutrophils through its A2a receptor (Ernens et al, supra). We have now found that administration of adenosine increases MMP-9 production by macrophages through its A3 receptor, in sharp contrast to our previous results obtained in neutrophils.

We therefore propose a novel therapeutic strategy to use an A3 antagonist (to inhibit MMP-9 production by macrophages) to treat patients with myocardial infarction or heart failure. We also propose a combination of A2a agonist (to inhibit MMP-9 secretion by neutrophils) and A3 antagonist (to inhibit MMP-9 production by macrophages).

### Results

### 1. Adenosine Increases MMP-9 Production by Primary Macrophages

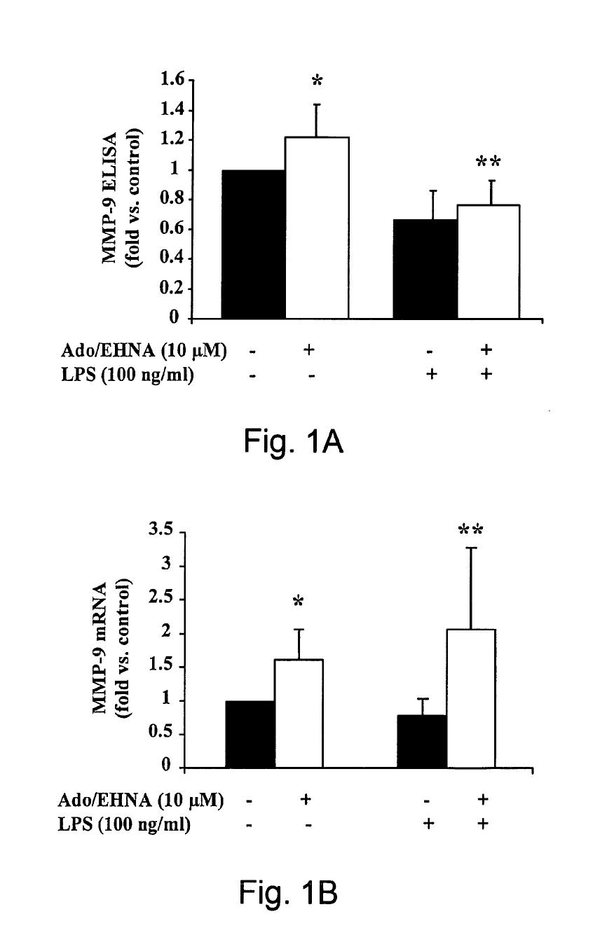

Monocytes were isolated from PBMCs obtained from healthy volunteers by negative selection and differentiated along the macrophage lineage by M-CSF. Using ELISA (FIG. 1A) and quantitative PCR (FIG. 1B), we detected a mild but significant increase of MMP-9 secretion in the conditions where adenosine together with the adenosine deaminase inhibitor EHNA (which reduces adenosine degradation) was added. This effect was reproduced when macrophages were activated by LPS. By gelatin zymography, we observed that two forms of MMP-9 were produced by macrophages: pro-MMP-9 homodimer and pro-MMP-9. In contrast to neutrophils, the MMP-9-lipocalin complex was not detected in macrophages. Furthermore, the active forms of MMP-9 and MMP-2 were not detected by zymography (not shown).

### 2. Adenosine Increases MMP-9 Production by THP-1-Derived Macrophages

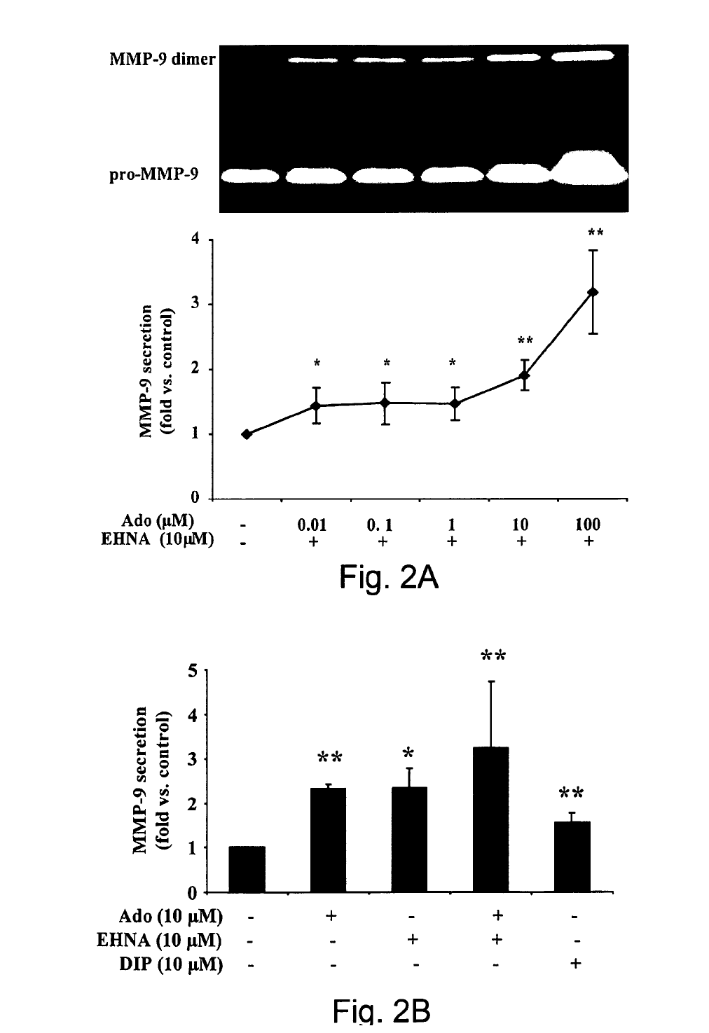

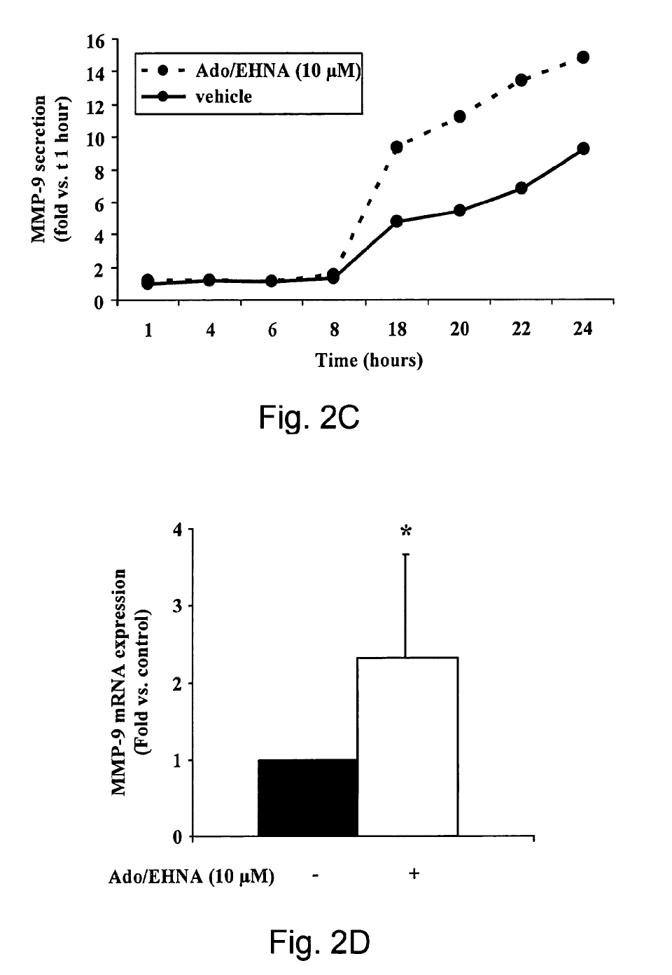

Having demonstrated that adenosine consistently increases MMP-9 secretion by primary macrophages, we explored the mechanisms responsible for this effect in macrophages differentiated from the monocytic cell line THP-1. Cells were incubated for 15 min with increasing concentrations of Ado and 10 μM EHNA before differentiation with 150 nM PMA for 48 hours. Adenosine increased MMP-9 production by macrophages in a concentration-dependent manner, reaching a 3-fold increase with 100 μM Ado (FIG. 2A lower panel). Concentrations of 10 μM Ado and 10 μM EHNA were used in further experiments. Considering that Ado concentrations found in vivo in conditions like heart failure and sepsis are in the micromolar range, our results suggest that the effect reported here is of biological relevance.

### 3. Both Endogenous and Exogenous Ado Enhance MMP-9 Secretion

To characterize the relative contribution of endogenous and exogenous Ado in MMP-9 production, we used several modulators of Ado metabolism. When added alone, Ado, EHNA, which enhances endogenous Ado concentration through inhibition of Ado deaminase, and DIP, which inhibits Ado transport, all triggered MMP-9 secretion. The effects of Ado and EHNA were additive since a combination of both drugs resulted in a higher MMP-9 secretion than each substance alone (FIG. 2B). These data show that both endogenous and exogenous Ado stimulate MMP-9 secretion and that their effects are additive.

4. Adenosine Increases MMP-9 mRNA Expression

To investigate the mechanism involved in enhanced MMP-9 secretion, we performed time-course experiments. An incubation period of at least 18 hours was necessary to reproduce the effect of Ado on MMP-9 (FIG. 2C). Using quantitative PCR, we observed that Ado induced a more than two-fold increase in MMP-9 mRNA expression in THP-1 cells which was slightly more than in primary macrophages (FIG. 2D). Overall, these observations suggest that the increase of MMP-9 secretion by Ado occurs through a transcriptional mechanism, in contrast to the degranulation process identified in neutrophils which is responsible for a high and rapid release of MMP-9.

### 5. Adenosine Increases MMP-9 Production Through its A3 Receptor

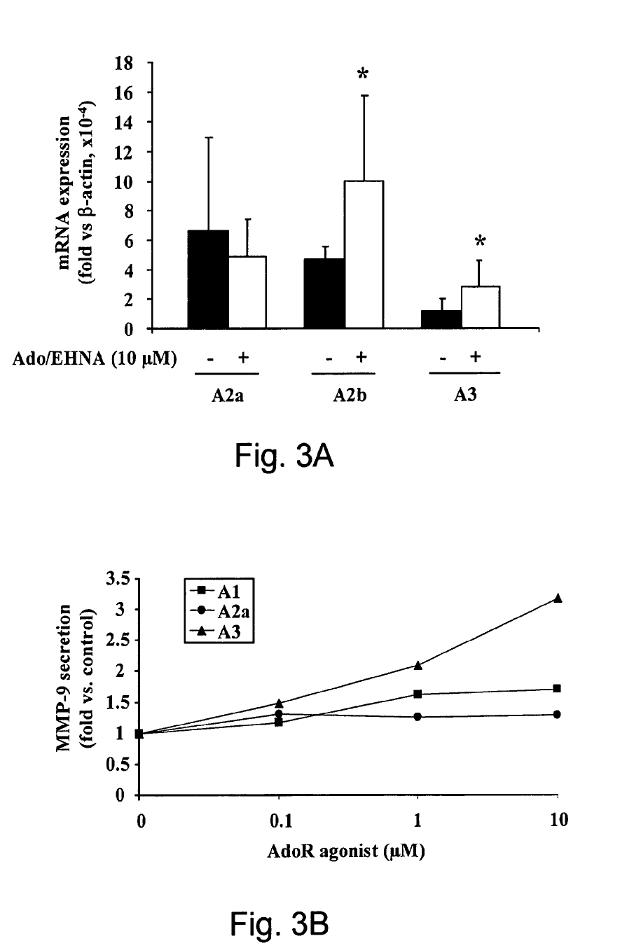

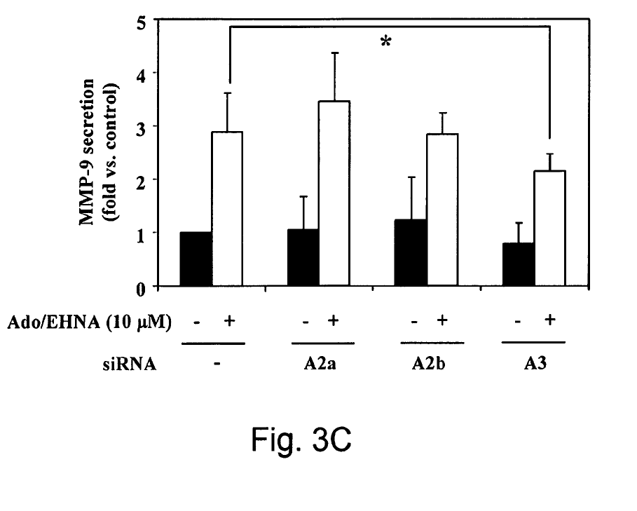

To address which type of receptor mediates the effect of Ado on MMP-9 secretion, we used both pharmacological and molecular approaches. First, we determined the relative expression of each of the four Ado receptors by macrophages. Quantitative PCR showed that receptors of the A2a type were the predominant form of Ado receptor in macrophages (FIG. 3A). No A1 receptor mRNA was detected in our experiments. Adenosine induced a more than two-fold increase of A3 and A2b expression. In experiments using agonists of Ado receptors, EHNA was omitted to specifically study the effect of exogenous Ado. FIG. 3B shows that only the A3-specific agonist IB-MECA was able to increase MMP-9 secretion to the same extent as Ado. Finally, a genomic approach using siRNA specific for A2a, A2b and A3 receptors was undertaken. For all experiments, we checked by quantitative PCR that each siRNA down-regulated the expression of its target receptor and not that of the other Ado receptors (not shown). Zymography revealed that only the A3-specific siRNA was able to significantly inhibit the Ado-mediated increase of MMP-9 secretion (FIG. 3C). Taken together, these results show that the A3 receptor mediates the effect of Ado on MMP-9 secretion.

### 6. Adenosine Improves Monocyte Migratory Capacity

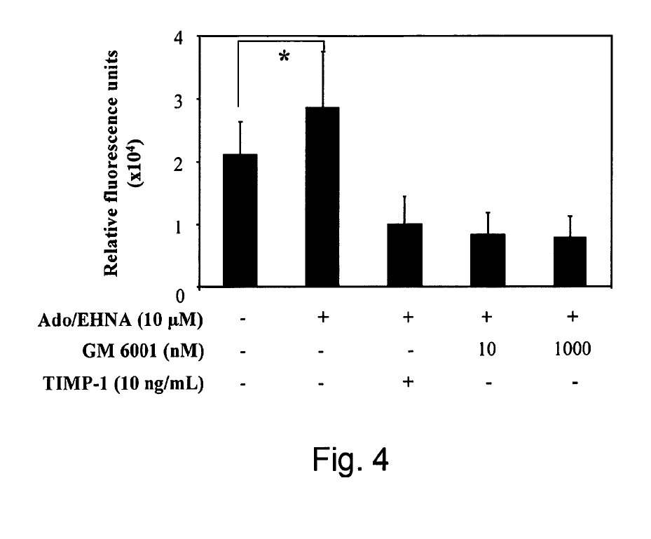

Infiltration of monocytes/macrophages into the myocardium is a hallmark of ventricular remodelling post MI. Cell migration is facilitated by degradation of the ECM by MMP-9. We therefore tested whether the increase in MMP-9 secretion by Ado could improve monocytes migration along a gradient of MCP-1. For this purpose, the top of a modified Boyden chamber was coated with gelatin B and MCP-1 was added in the bottom compartment. Monocytes were treated with Ado before seeding on the microporous membrane of the chamber. As shown in FIG. 4, Ado enhanced cell migration through the gelatin layer. This effect was inhibited by the synthetic MMP inhibitor GM6001 and the endogenous MMP-9 inhibitor TIMP-1 (FIG. 4). Together, these results demonstrate that Ado enhances monocytes/macrophages migration through increased MMP-9 activity.

### FIGURE LEGENDS

FIG. 1. Adenosine increases MMP-9 production by primary macrophages. Monocytes isolated from PBMCs of healthy volunteers by negative selection were differentiated with 50 ng/ml M-CSF for 7 days. Macrophages were incubated for 15 minutes with Ado and EHNA (10 μmol/L each) or vehicle, then LPS (100 ng/ml) or vehicle was added and cells were incubated for another 24 hours before harvesting. A. MMP-9 secretion in cell supernatant as measured by ELISA was significantly increased by Ado, whether macrophages were treated with LPS or not. B. Quantitative PCR revealed that Ado also increased MMP-9 mRNA expression. Results are mean±SD (n=12 for A, n=8 for B). * P<0.05 vs control (macrophages treated with Ado/EHNA vehicle), ** P<0.01 vs LPS.

FIG. 2. Adenosine increases MMP-9 production by THP-1-derived macrophages. THP-1 cells were treated for 15 min with Ado and 10 μM EHNA or vehicle before differentiation to macrophages with 150 nM PMA for 48 hours. EHNA, which inhibits Ado deaminase, and DIP, which inhibits Ado transport, were used to enhance endogenous Ado concentration. Gelatinase activity was measured in cell-free conditioned medium by zymography and densitometry. A. A representative zymogram is shown. Densitometric analysis revealed that Ado concentration-dependently increased MMP-9 secretion by macrophages. B. Zymography showed that both endogenous and exogenous Ado enhanced MMP-9 secretion. Alone, Ado and EHNA triggered MMP-9 secretion and their effects were additive. DIP also increased MMP-9 secretion. C. Time-course experiment. Measuring MMP-9 release in culture medium by zymography revealed that a differentiation period of 18 hours was necessary to trigger MMP-9 secretion and that the effect of Ado was seen also from 18 hours. D. The increase of MMP-9 involves a transcriptional mechanism. Quantitative PCR showed that Ado increased MMP-9 mRNA expression after 48 hours of differentiation. Results are mean±SD (n=4 for A and B, n=1 for C, n=6 for D). * P<0.05 vs control (macrophages treated with Ado/EHNA vehicle), ** P<0.01 vs control.

FIG. 3. Adenosine increases MMP-9 production through its A3 receptor. A. THP-1 cells were treated for 15 min with 10 μM Ado and 10 μM EHNA or vehicle before differentiation to macrophages with 150 nM PMA for 48 hours. Quantitative PCR revealed that A2a and A2b were the predominant forms of Ado receptors in macrophages. Adenosine induced a more than two-fold increase of A3 and A2b expression. Results are mean±SD (n=6). * P<0.05 vs control (macrophages treated with Ado/EHNA vehicle). B. THP-1 cells were treated for 15 min with 0.1, 1 or 10 μM of Ado receptors agonists (CPA, A1-specific; CGS21680, A2a-specific; IB-MECA, A3-specific). Subsequently, differentiation was achieved with 150 nM PMA for 48 hours. Gelatinase activity was assessed in conditioned medium. The experiment was performed twice with similar results. C. The A3-specific siRNA inhibits the Ado-mediated increase of MMP-9 secretion. Cells were transfected with siRNA specific for A2a, A2b or A3 Ado receptors, incubated for 48 hours to achieve down-regulation of Ado receptors expression, treated for 15 min with 10 μM Ado and 10 μM EHNA or vehicle before differentiation to macrophages with 150 nM PMA for 48 hours. Conditioned medium was analysed by gelatin zymography. Results are mean±SD (n=3). * P<0.05 vs Ado (macrophages treated with Ado/EHNA).

FIG. 4. Ado increases monocytes migratory capacity. THP-1 monocytes were treated for 24 hours with 10 μM Ado and 10 μM EHNA or vehicle before seeding on the microporous membrane of a modified Boyden chamber pre-coated with gelatin B. 10 ng/mL MCP-1 was added in the bottom compartment. When specified, cells were pre-incubated with TIMP-1 (10 ng/mL) or GM6001 (10 nM or 1 μM) before seeding on the membrane. Cell migration was quantified by fluorescence after 24 hours. Ado enhanced cell migration and TIMP-1 or GM6001 partly prevented this effect. Results are mean±SD (n=5). * P<0.05 vs no Ado.

### Methods

Materials. All materials and reagents were from Sigma (Bornem, Belgium) unless specified otherwise. Ado receptor agonists were CPA (C8031, N6-CyclopentylAdo, A1 specific), CGS21680 (C141, 2-[4-[(2-carboxyethyl)phenyl]ethylamino]-5′-N-ethylcarbamoyl, A2a specific), IB-MECA, (1-deoxy-1-[6-[[(3-iodophenyl)methyl]amino]-9H-purine-9-yl]-N-methyl-Dribofuranuronamide, A3 specific). Small interfering RNAs (siRNAs) were purchased from Qiagen (Hilden, Germany). GM 6001 ((R)-N-4-Hydroxy-N1-[(S)-2-(1H-indol-3-yl)-1-methylcarbamoly-ethyl]-2-isobutyl-succinamide) and the recombinant human protein TIMP-1 (R&D Systems, Abingdon, U.K.) were used as MMPs inhibitors. Macrophage-colony stimulating factor (M-CSF) and MCP-1 were purchased from Peprotech (Levallois-Peret, France). Endotoxin contamination of all chemicals used was below the detection limit of the limulus amebocyte lysate test (0.05 EU/mL, E-Toxate® Kit, Sigma). Absence of cytotoxicity of each treatment was checked by measuring the release of the cytoplasmic enzyme lactate dehydrogenase using the Cytotoxicity Detection Kit (Roche Diagnostics, Mannheim, Germany) according to the manufacturer's instructions.

Cell culture. All cell culture reagents were from Lonza (Verviers, Belgium) unless specified otherwise. Peripheral venous blood was obtained from healthy volunteers. Peripheral blood mononuclear cells were obtained by Ficoll gradient. Monocytes were purified by negative selection using the Monocyte Isolation Kit II (Myltenyi Biotec GmbH, Bergisch Gladbach, Germany). Differentiation was achieved with 50 ng/mL M-CSF for 7 days. Macrophages were incubated for 15 minutes with Ado/EHNA (10 μmol/L) or vehicle, then LPS (100 ng/ml) or vehicle was added and cells were incubated for another 24 hours before harvesting. Cells from the monocyte-like line THP-1 (ATCC, LGC Promochem, Teddington, UK) were propagated at 37° C.-5% CO₂ in RPMI 1640 medium supplemented with L-glutamine (2 mM), Penicillin (100 Units/ml), Streptomycin (100 μg/ml), non-essential amino acids solution (1 μM), sodium pyruvate (1 mM), and 10% heat-inactivated fetal calf serum (FCS) (Eurobio, Les Ulys, France). For experiments, FCS in culture medium was reduced to 1%. Cells were treated for 15 min with EHNA vehicle (DMSO), Ado (0.01 to 100 μM), Ado receptors agonists (0.1 to 10 μM), EHNA (10 μM) or Dipyridamole (DIP) (10 μM). Cells were then differentiated into macrophages with 150 nM phorbol myristate actetate (PMA) for 24 hours. Macrophages were subsequently treated with LPS (1 to 1000 ng/ml), fMLP (10⁻⁷ M) or H₂O₂ (10 mM) for another 24 hours.

Transfection was achieved using the Nucleofection technology and the Nucleofector™ solution V according to the manufacturer's instructions (Amaxa Inc., Cologne, Germany). Forty eight hours before Ado treatment, cells were transfected with 1.4 μg siRNA specific for each Ado receptor.

Selected target mRNA sequences for Ado receptors are the following:

| | |
|---|---|
| siRNA A2a: | |
| 5′-CAGGAGTGTCCTGATGATTCA-3′; | (SEQ ID NO 6) |
| siRNA A2b: | |
| 5′-CACGTATCTAGCTAATATGTA-3′; | (SEQ ID NO 7) |
| siRNA A3: | |
| 5′-CCCTATCGTCTATGCCTATAA-3′. | (SEQ ID NO 8) |

Cells were lysed in TriReagent® for RNA harvesting. Cell-free conditionned medium was collected, mixed with protease inhibitors (Roche, Mannheim, Germany) and Bovine Serum Albumin (BSA, 0.2% final concentration) for further ELISA or zymography. All samples were stored at—80° C. until analysis.

Real time quantitative PCR. Total RNA was isolated using TriReagent® and the RNeasy mini kit (Qiagen, Hilden, Germany) according to the manufacturer's instructions. Potential contaminating genomic DNA was digested by DNase I treatment (Qiagen). One microgram of total RNA was reverse-transcribed using the Superscript® II Reverse Transcriptase (Invitrogen, Merelbeke, Belgium). PCR primers were designed using the Beacon Designer software (Premier Biosoft, Palo Alto, USA) and were chosen to encompass an intron. PCR was performed using the iCycler® and the IQ™ SYBR® Green Supermix (Biorad, Nazareth, Belgium). 1/10 dilutions of cDNA were used. PCR conditions were as follows: 3 mM at 95° C., 30 sec at 95° C. and 1 min annealing (40 cycles). Melting point analysis was obtained after 80 cycles for 10 sec from 55° C. up to 95° C. Each run included negative reaction controls. β-actin was chosen as housekeeping gene for normalization. Expression levels were calculated by the relative quantification method (ΔΔCt) using the Genex software (Biorad, Nazareth, Belgium) which takes into account primer pair efficiency.

Analysis of gelatinase activity. Gelatin zymography was performed on culture supernatants to assess secreted MMP-9 activity. Briefly, conditioned medium was loaded on SDS-polyacrylamide gels containing 0.2% gelatin under non reducing conditions. After electrophoresis, gels were washed and incubated overnight at 37° C. in assay buffer (50 mM Tris-HCl, pH 7.6, 200 mM NaCl, 5 mM CaCl₂, and 0.02% Brij35). Subsequently, gels were stained in 0.1% Coomassie Blue and destained in 25% ethanol/8% acetic acid. Densitometry was achieved using the Gel Logic 2200 Digital Imaging system and Aida Software (Kodak, Zavemtem, Belgium).

ELISA. Total MMP-9 and TIMP-1 concentrations in conditioned medium were measured by ELISA (R&D Systems, Abingdon, U.K.). Detection limits were 0.156 ng/mL for MMP-9 and 0.08 ng/mL for TIMP-1.

Migration assay. Migratory capacity of THP-1 monocytes was studied using a Transwell® system with polycarbonate microporous membranes (5 μm pore size, 24-well chamber, Costar, Lowell, USA). Membranes were coated with a 2% gelatin B solution and allowed to dry at room temperature for 2 hours. MCP-1 (10 ng/mL) was added in the bottom compartment. THP-1 monocytes were cultured in serum- and antibiotic-free medium with or without 10 μM Ado and 10 μM EHNA for 24 hours. Cells were pre-incubated for 30 min with GM 6001 (10 nM or 1 μM) or TIMP-1 (10 ng/mL) before seeding into the upper compartment at a concentration of 7.5×10⁴ cells per well. After 24 hours, cells that migrated through the membrane were detached, lysed and stained by the CyQuant GR® dye (Invitrogen) for 15 min. Fluorescence was read with a POLARstar Optima (BMG LABTECH, Champigny-sur-Marne, France) microplate reader (Xex=492 nm, kem=520 nm).

Statistical analysis. Results are expressed as mean±S.D. Data with a Gaussian distribution were analyzed by paired t-test. Mann-Whitney (unpaired data) or Wilcoxon (paired data) tests were used for non-Gaussian data. A P value <0.05 was considered significant.

1. A method of treatment or prophylaxis of a disease or condition in a patient, which disease comprises congestive heart failure and/or a disease or condition associated with congestive heart failure, the method comprising administering an adenosine A3 receptor antagonist to the patient.

2. The method of claim 1, wherein the disease or condition comprises myocardial infarction, acute coronary syndrome, ischaemic cardiomyopathy, non-ischaemic cardiomyopathy, acute heart failure, or chronic heart failure.

3. The method of claim 1, wherein treatment or prophylaxis comprises decreasing levels of matrix metalloproteinases in the patient, which patient has myocardial infarction or heart failure.

4. The method of claim 1 wherein treatment or prophylaxis comprises inhibiting the development of ventricular remodelling and heart failure after myocardial infarction.

5. The method of claim 4 wherein treatment or prophylaxis comprises inhibiting maladaptive remodelling of the myocardium.

6. The method of claim 1, wherein the adenosine A3 receptor antagonist is selected from the group consisting of: MRS1067, MRS1097, L-249313, L-268605, CGS15943, KF26777, MRS1220, MRS1523, and PSB10.

7. The method of claim 1, wherein treatment or prophylaxis further comprises administering one or more additional adenosine agonists or antagonists.

8. The method of claim 7, wherein the one or more additional adenosine agonists or antagonists comprise an adenosine A2a receptor agonist.

9. The method of claim 8, wherein a molecule having both A3 receptor antagonist activity and A2a receptor agonist activity is administered.

10. The method of claim 9, wherein the molecule is (2R,3R,4S,5R)-2-(6-amino-2-{[(1S)-2-hydroxy-1-(phenylmethyl)ethyl]amino}-9H-purin-9-yl)-5-(2-ethyl-2H-tetrazol-5-yl)tetrahydro-3,4-furandiol.

11. The method of claim 1, wherein the sequence of the adenosine A3 receptor comprises the sequence of SEQ ID NO 1, or a variant having at least 75% sequence homology therewith, or a corresponding sequence capable of hybridizing to said SEQ ID NO 1 under highly stringent conditions of 6×SSC.

12. The method of claim 1, wherein the adenosine A3 receptor antagonist is capable of reducing the levels of a matrix metalloproteinase (MMP) in the blood and in and around the heart.

13. The method of claim 12, wherein the MMP is MMP-9 and/or wherein the MMP comprises the protein sequence set out in SEQ ID NO 3.

14. The method of claim 1, wherein the A3 antagonist is selective.

15. (canceled)

16. The method of claim 8, wherein the adenosine A2a receptor agonist is adenosine, an adenosine agonist, or an adenosine analogue.

17. The method of claim 16, wherein the A2a agonist is Regadenoson (CVT-3146), CGS 21680, APEC or 2HE-NECA.

18. (canceled)

19. A method of treating a patient having myocardial infarction or heart failure, the method comprising administering an adenosine A3 receptor antagonist and/or administering one or more additional adenosine agonist or adenosine antagonist.

20. A method for lowering or reducing MMP-9 levels in a patient's heart or for the treatment or prophylaxis of heart failure which treatment or prophylaxis is correlated with lower MMP-9 levels, the method comprising administering an adenosine A3 receptor antagonist and/or an adenosine A2a receptor agonist.

21. A method of preventing degradation of myocardial tissue associated with myocardial infarction or acute heart failure, the method comprising administering an adenosine A3 receptor antagonist and/or administering one or more additional adenosine agonist or adenosine antagonist.

22. A method of preventing degradation of myocardial tissue associated with end-stage heart disease, the method comprising administering an adenosine A3 receptor antagonist and/or administering an adenosine A2a receptor agonist.

23. A method of treating a patient presenting symptoms of congestive heart failure, the method comprising administering an agent which decreases the production of matrix metalloproteinases in the patient's myocardial tissue, wherein the agent is an adenosine A3 receptor antagonist.

24. The method of claim 23, wherein the condition is myocardial infarction, acute coronary syndrome, ischaemic cardiomyopathy, non-ischaemic cardiomyopathy, acute heart failure, or chronic heart failure.

25. The method of claim 21 or 23, wherein an adenosine A2a receptor agonist is also administered.

26. A method of treatment or prophylaxis of a disease or condition associated with congestive heart failure, the method comprising administering one or more antisense polynucleotides to a patient or administering gene suppression to a patient, which antisense polynucleotides or gene suppression are targeted to an adenosine A3 receptor (A3AR) or are capable of reducing the level of expression of said receptor.
